(19) United States
(12) Patent Application Publication
(10) Pub. No.: US 2020/0263117 A1
(43) Pub. Date: Aug. 20, 2020

(54) Process for Improving Protein Recovery in Stillage Processing Streams

(71) Applicant: Ecolab USA Inc., St. Paul, MN (US)

(72) Inventor: Lan Xiao, Naperville, IL (US)

(73) Assignee: Ecolab USA Inc., St. Paul, MN (US)

(21) Appl. No.: 16/791,841

(22) Filed: Feb. 14, 2020

Related U.S. Application Data
(60) Provisional application No. 62/806,481, filed on Feb. 15, 2019.

(51) Int. Cl. C12F 3/10; C07K 1/30

(52) U.S. Cl. CPC C12F 3/10; B01D 21/01; C07K 1/30

(57) ABSTRACT

Provided is a method of processing stillage from an ethanol production process. The method comprises treating stillage comprising oil, protein, and water upstream of a separation, concentration or evaporation step with at least one coagulant and at least one flocculant, thereby forming treated thin stillage comprising solids which include at least a portion of the oil and protein; and clarifying the treated stillage via a solid/liquid separation process thereby forming clarified stillage and a separated solids phase comprising at least a portion of the solids from the treated stillage.

### CROSS-REFERENCE TO RELATED APPLICATIONS

This patent application claims the benefit of U.S. Provisional Patent Application No. 62/806,481, filed Feb. 15, 2019, the disclosure of which is incorporated herein by reference for all purposes.

### BACKGROUND OF THE INVENTION

Stillage process streams typically involve milling/grinding, further processing, separation, and recovery/separation of solids and oils from the stillage. For example, in a dry milling process for the manufacture of ethanol, corn is ground up and processed to produce a “beer mash” which is fermented to form ethanol. Once the stream reaches the desired ethanol content, the material is then transferred to a stripper column. The stripper column facilitates recovery and removal of the ethanol and the remaining stream, known as whole stillage, is passed on for further processing.

In ethanol production processes that involve dry milling of corn, for example, whole stillage contains non-fermentable components of the corn kernels including germ, protein, gluten, and fiber, as well as fats and oils and a small amount of starch, in addition to dead yeast cells. Whole stillage typically contains 9%-14% total solids of which 4% to 10% are suspended solids and 4% to 5% are dissolved solids. Many of the components of whole stillage; i.e., oil and protein solids, are useful, and considerable attention has been devoted in the industry to develop methods to separate and recover those components.

Typically, various uses of heat and centrifuge pressures are applied to whole stillage, thin stillage, or syrup to recover at least some of these components. Typical prior art processes involve centrifuging away water from the whole stillage thereby forming a wet cake of concentrated solids and a thin stillage stream that is low in solids. The thin stillage then undergoes some form of drying or evaporation to form a viscous syrup. Part of the thin stillage stream may be reused in the process by recirculating to the front of the plant as backset and mixing it with new corn. The syrup is typically added to other solids recovered from the process to form a mass commonly known as Distiller Dry Grains and Solubles (DDGS), which can be used, for example, as an animal feed.

U.S. Pat. Nos. 9,051,538 and 9,516,891 disclose a multi-stage process for the separation of bio-components from a waste stream containing DDGS, in which the waste stream is separated into a stream containing predominantly protein, a stream containing predominantly oil, a stream containing predominantly water, and a stream that contains predominantly fibers, by using polymers and separation equipment including a plate separator, a press and a dissolved air floatation device. U.S. Pat. No. 7,497,955 discloses a method of dewatering thin stillage process streams by adding to the process streams a flocculating amount of an anionic copolymer comprising a monomer unit derived from acrylic acid. U.S. Pat. No. 9,776,105 discloses a method of treating thin stillage upstream of a concentration or evaporation step with an inverse emulsion containing an anionic flocculant and an emulsifying agent.

There is a clear need and utility for improved methods, systems, and apparatus for improving clarification of ethanol stillage and generating higher protein DDGS product, while maintaining or improving oil recovery from the stillage.

### BRIEF SUMMARY OF THE INVENTION

The invention provides a method of processing stillage from an ethanol production process, which method involves treating stillage comprising oil, protein, and water upstream of a separation, concentration or evaporation step with at least one coagulant and at least one flocculant, to produce a treated stillage comprising solids which include at least a portion of the oil and protein; and clarifying the treated stillage via a solid/liquid separation process, to produce a clarified stillage comprising a clarified aqueous phase and a separated solids phase, wherein the separated solids phase comprises at least a portion of the solids from the treated stillage. In some embodiments, the separated solids phase may be in the form of a float layer, e.g., obtained in a process for producing ethanol from dry milled/dry ground corn.

The method of the invention can be applied to any suitable stillage process for producing ethanol. For example, the method of the invention may be applied to stillage processes in which the ethanol is produced in an ethanol biofuel plant, a spirits distillery, or a brewery or the like. The method of the invention may be applied in ethanol production processes that use a wet milling process or a dry grind process.

The method of the invention may be used in the treatment of either whole stillage or thin stillage.

In the method of the invention, the coagulant may include, e.g., one or more inorganic coagulants, or a blend of one or more inorganic coagulants and one or more organic coagulants. The flocculant may include, e.g., an anionic flocculant. The solid/liquid separation process may include, e.g., dissolved air flotation, induced air flotation, or a combination thereof.

In some embodiments, the method of the invention further includes separating at least a portion of the oil from the separated solids phase, e.g., a float layer, to produce a de-oiled separated solids phase, e.g., a de-oiled float layer. The de-oiled separated solids phase produced according to the invention may be processed further, e.g., by drying and/or other treatment methods, to produce dry grains containing protein such as, e.g., distiller dry grain containing protein.

The present invention further provides an ethanol production process which includes the present inventive method of processing stillage produced therein.

The present invention also provides dried and/or dry grains, e.g., distiller dry grain produced according to the method of the invention, and compositions containing the dried/dry grains, e.g., distiller dry grains. The invention further provides a nutritional product comprising the dried/dry grains of the invention, e.g., distiller dry grain, produced according to the method of the invention, as well as a livestock feed or fertilizer comprising the dried/dry grains of the invention, e.g., distiller dry grain produced according to the method of the invention. The invention further provides livestock feed or fertilizer which may further comprise biological sludge or other nutrients.

### BRIEF DESCRIPTION OF THE SEVERAL VIEWS OF THE DRAWING(S)

FIG. 1 depicts a flowchart illustrating a conventional method of processing stillage in a dry grind biofuel ethanol production process.

FIG. 2 depicts a flowchart illustrating a conventional method of processing stillage in a spirits distillery, e.g., a scotch whisky production process.

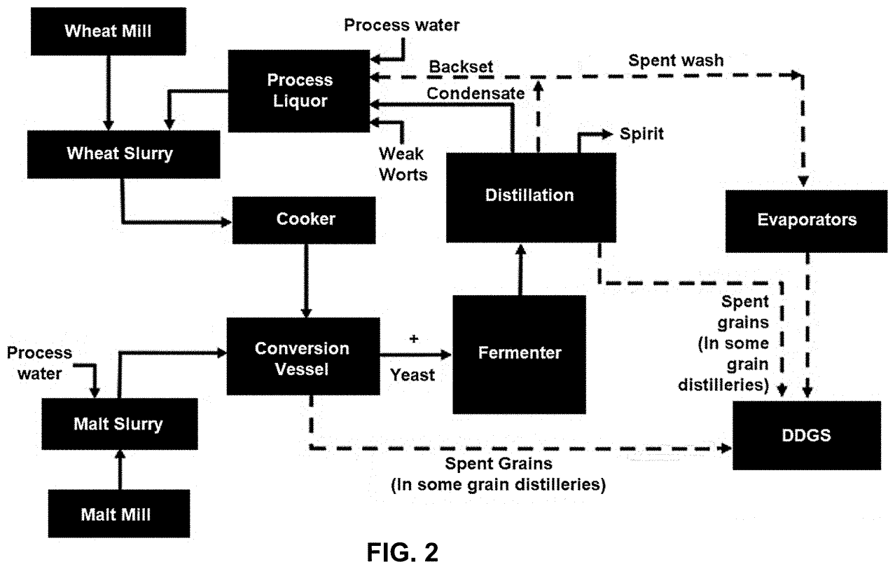

FIG. 3 depicts a flowchart illustrating a conventional method of processing stillage in a brewery production process.

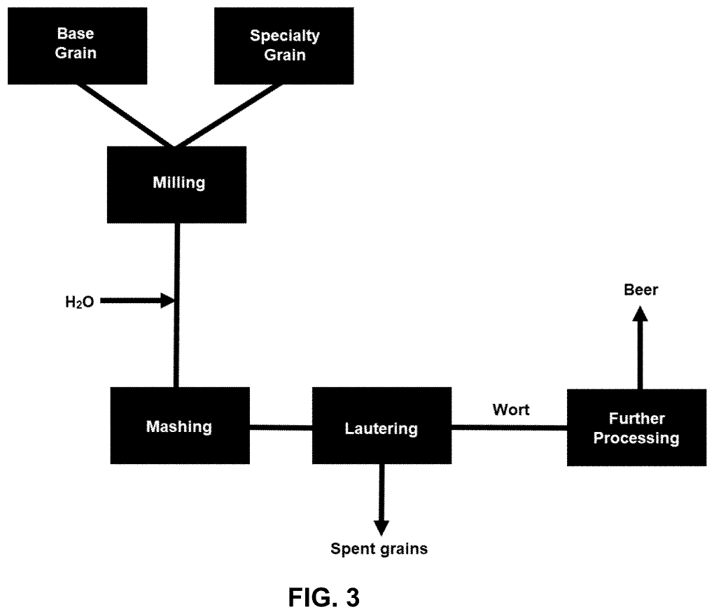

FIG. 4 depicts a flowchart illustrating one manner of implementing the method of the present invention in a dry grind biofuel ethanol production process.

FIG. 5 depicts a graph illustrating protein recovery dosage curves in the solid phase after samples of thin stillage were treated with a fixed amount of the same anionic flocculant and the indicated coagulant in lab simulations.

FIG. 6 depicts a graph showing maximum percent protein recovery in the solid phase for each coagulant tested depicted in FIG. 5, as well as the indicated control samples.

FIG. 7 depicts a graph illustrating maximum percent protein recovery in the solid phase from thin stillage treated with different coagulants in a comparable simulation, as well as the indicated control samples.

FIG. 8 depicts a graph illustrating a protein recovery dosage curve from the GEM float obtained in an ethanol biofuel plant.

### DETAILED DESCRIPTION OF THE INVENTION

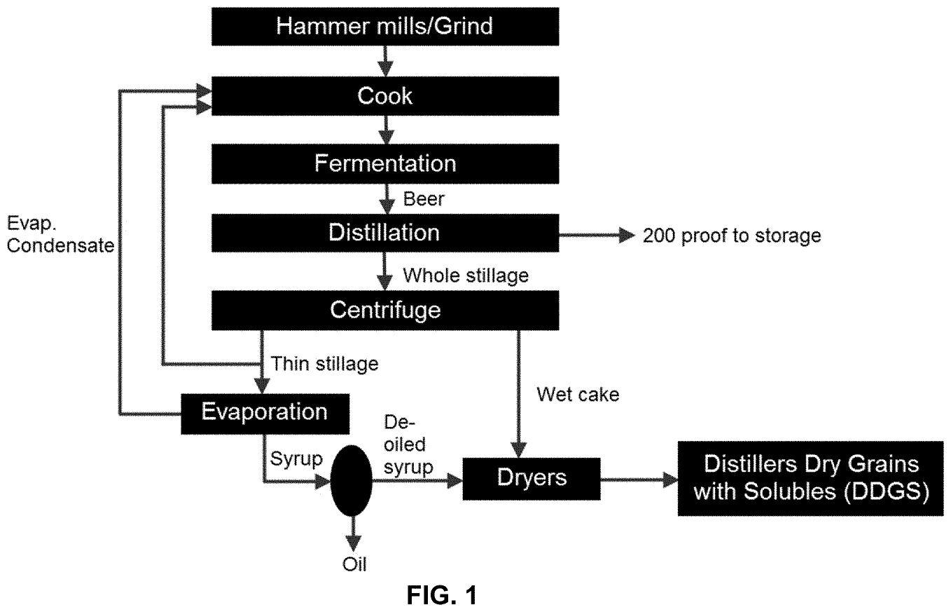

As illustrated in FIG. 1, conventional processing requirements using mechanical means to extract the various products of a dry milling stillage process stream has disadvantages, among them high capital costs and high energy costs. In addition, the amount of the various products (protein, oil) that can conventionally be recovered from stillage with mechanical means is limited.

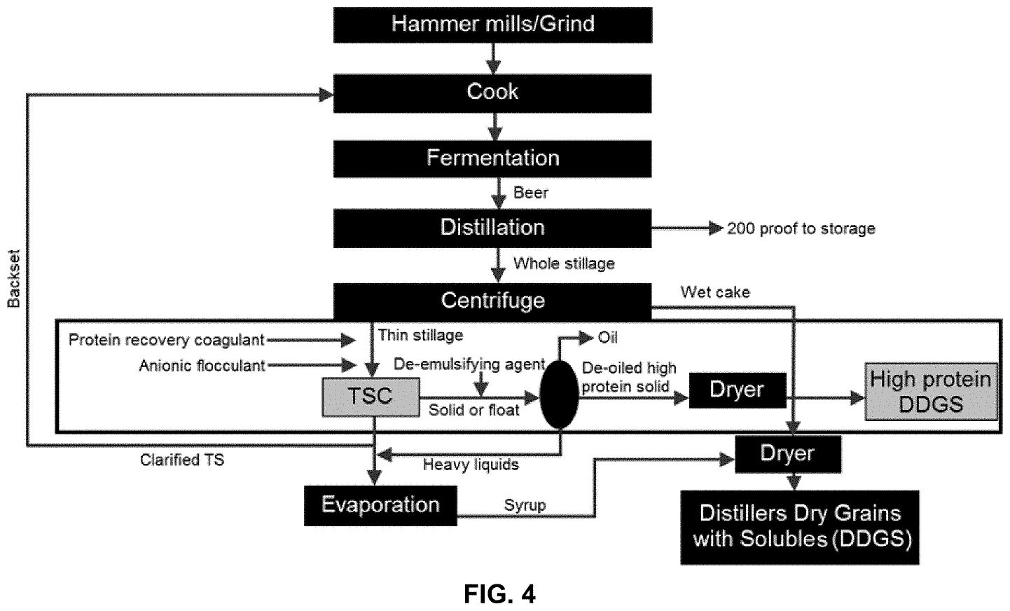

The present invention provides an improved method of processing stillage from an ethanol production process, which includes treating stillage comprising oil, protein, and water upstream of a separation, concentration or evaporation step with at least one coagulant and at least one flocculant, to produce a treated stillage containing solids (e.g., coagulated and/or flocculated solids) which include at least a portion of the oil and protein; and clarifying the treated stillage via a solid/liquid separation process, to produce a clarified stillage containing a clarified aqueous phase and a separated solids phase, which in some embodiments may be in the form of a float layer, comprising at least a portion of the solids. A flowchart illustrating one manner of implementing the inventive method in a dry grind biofuel process is shown in FIG. 4.

In some embodiments, the method of the invention may be used in processing whole stillage, thin stillage, or a combination thereof in a dry grind biofuel process or a spirits distillery. In one embodiment, the method of the present invention is used for processing thin stillage.

In some embodiments, the method of the invention further includes separating at least a portion of the oil from the float layer obtained in a dry grind biofuel process to produce a de-oiled float layer. In accordance with some embodiments, the de-oiled separated solids phase, e.g., the de-oiled float layer, may be further processed, e.g., by drying and/or other treatment methods, to produce dry grains comprising protein, e.g., distiller dry grain comprising protein. The method of the present invention surprisingly provides distiller dry grains with an enriched protein content relative to the DDG produced according to conventional methods that utilize flocculant without a coagulant.

The coagulant preferably includes one or more chemical species that induce coagulation, i.e., the initial agglomeration of material suspended within a liquid. In the method of the invention, the coagulant may include one or more inorganic coagulants. The inorganic coagulants may be cationic, such as trivalent or divalent metal salts having counterions including sulfate, chloride, phosphorous, or hydroxy chloride. The inorganic cationic coagulants may be ferric-based, aluminum-based, or a combination thereof. In some embodiments, the inorganic coagulant may include aluminum sulfate, poly-aluminum chloride, aluminum chlorohydrate, sodium aluminate, ferric sulfate, ferric chloride, or ferrous sulfate, or a combination thereof. In some embodiments, the inorganic coagulant includes aluminum chlorohydrate, poly-aluminum chloride, ferric sulfate, ferric chloride, or a combination thereof, some of which are commercially available from Nalco Company, Naperville, Ill. as Ultrion 8187, Ultrion 8117, and Ferralyte 8131.

In some embodiments, the coagulant includes a blend of one or more inorganic coagulants and one or more organic coagulants. The organic coagulant may include one or more water-soluble polyelectrolytes or amine-based polyelectrolytes. Examples of suitable organic coagulants include poly(diallyldimethylammonium chloride) (polyDADMAC), epichlorohydrin-diethylamine, dimethylamine, polyamines, polyquaternary amines, or a combination thereof. In some embodiments, the organic coagulants are poly(diallyldimethylammonium chloride) (polyDADMAC), epichlorohydrin-diethylamine, or a combination thereof. These coagulants may be obtained commercially from Nalco Company, Naperville, Ill. as GR-308 and GR-305, respectively.

In some embodiments, a preferred coagulant includes a blend of ferric chloride and epichlorohydrin-diethylamine available commercially from Nalco Company, Naperville, Ill. as Cat-Floc 71264.

Generally, the coagulant may be added to the stillage process stream at a dosage sufficient to provide concentration of coagulant in the stillage of about 10 to about 1,000 ppm, e.g., at a dosage of about 50 ppm to about 1,000 ppm, at a dosage of about 100 ppm to about 1,000 ppm, at a dosage of about 200 ppm to about 1,000 ppm, at a dosage of about 500 ppm to about 1,000 ppm, at a dosage of about 50 ppm to about 100 ppm, or at a dosage of about 50 ppm to about 500 ppm.

In accordance with the present invention, if desired, coagulation and/or settling can be aided by the use of microparticulates. “Microparticulates” generally refer to certain insoluble materials which may be added to the process stream to physically interact with the suspended solids, fats, oils and/or greases in the process stream in such a way as to facilitate the separation and removal of these components by physical interaction. Without being bound by any particular theory, it is believed that addition of these materials provides a surface area and sites where polymers can interact and bridge the suspended particles forming an agglomerated particle or a floc. The use of microparticles may result in a floc or agglomerated particle that is more resistant to mechanical shear and as a result may use a physical sweep floc mechanism to capture and remove suspended solids, fats, oils and greases from the water phase. Once the desired particle interactions are achieved, the microparticulates may facilitate the separation process by increasing the rate of solids settling. Representative microparticulates may include, e.g., bentonite clay, montmorillonite clay, particularly montmorillonite clay available from CETCO, Arlington Heights, Ill. under the tradename AltaFloc, microsand (80 mesh silica sand), colloidal silica, colloidal borosilicate, starch and the like, and combinations thereof.

“Colloidal silica” and “colloidal borosilicate” generally refer to a stable aqueous dispersion of silica particles, e.g., amorphous silica particles or borosilicate particles, e.g., amorphous borosilicate particles, respectively, having a suitable particle size, e.g., having a particle size of up to about 500 μm, e.g., up to about 100 μm, up to about 50 μm, up to about 10 μm, up to about 1 μm, up to about 500 nm, or up to about 100 nm. Colloidal silica and colloidal borosilicate may be manufactured from known materials such as sodium silicate or borosilicate and are commercially available, for example from Nalco Company, Naperville, Ill.

Examples of suitable microparticulates include bentonite, montmorillonite, microsand, colloidal silica and colloidal borosilicate, and combinations thereof.

The microparticulate may be added to the stillage process stream prior to or after addition of the any coagulant(s) or flocculant(s), e.g., at a dosage sufficient to provide a concentration of microparticles in the stillage of about 10 to about 1,000 ppm.

The flocculant may include one or more chemical species which induces flocculation, e.g., by enhancing agglomeration of material suspended within a liquid either alone or after coagulation when the liquid is stirred or otherwise mixed. In the method of the invention, the flocculant may include at least one anionic flocculant. The anionic flocculant preferably creates a concentrated solids layer containing oil and insoluble protein. This concentrated layer in turn may be separated using known oil/solid/water mechanical separation techniques such as decanter, tricanter and stacked disk centrifuges. In some embodiments of the invention, the mechanical processing is performed with a stacked disk centrifuge.

Anionic polymers suitable for use in the method of this invention may include, for example, polymers prepared by polymerizing acrylic acid sodium salt, methacrylic acid sodium salt, 2-acrylamido-2-methyl-1-propanesulfonic acid sodium salt, or a combination thereof, and optionally one or more acrylamide monomers, under free radical forming conditions using methods known in the art of polymer synthesis. Such anionic polymers are commercially available, for example from Nalco Company, Naperville, Ill.

In some embodiments, the anionic polymer is cross-linked with about 0.005 to about 10 ppm of one or more cross linking agents. Representative cross-linking agents include but are not limited to N,N-methylenebisacrylamide, N,N-methylenebismethacrylamide, triallylamine, triallyl ammonium salts, ethylene glycol dimethacrylate, diethylene glycol dimethacrylate, polyethylene glycol diacrylate, triethylene glycol dimethylacrylate, polyethylene glycol dimethacrylate, N-vinylacrylamide, N-methylallylacrylamide, glycidyl acrylate, acrolein, glyoxal, vinyltrialkoxysilanes and the like. In some embodiments, the cross-linking agent includes from N,N-methylenebisacrylamide, polydiethyleneglycoldimethacrylate, trimethylolpropane ethoxylate (x EO/y OH) tri(meth)acrylate, where x=1-20 and y=1-5, trimethylolpropane propoxylate (x EO/y OH) triacrylate, where x=1-3 and y=1-3, 2-hydroxyethylmethacrylate, or a combination thereof.

In some embodiments, the anionic polymer includes one or more of: dry polymers, emulsion polymers, inverse emulsion polymers, latex polymers, dispersion polymers, and mixtures thereof. The advantages of polymerizing water-soluble monomers as inverse emulsions include 1) low fluid viscosity can be maintained throughout the polymerization, permitting effective mixing and heat removal, 2) the products can be pumped, stored, and used easily since the products remain liquids, and 3) the polymer “actives” or “solids” level can be increased dramatically over simple solution polymers, which, for the high molecular weight flocculants, are limited to lower actives because of viscosity considerations. The inverse emulsion polymers may then be “inverted” or activated for use by releasing the polymer from the particles using shear, dilution, and, generally, another surfactant, which may or may not be a component of the inverse emulsion.

The inverse emulsion polymer may be prepared by dissolving the desired monomers in an aqueous phase, dissolving the emulsifying agent(s) in an oil phase, emulsifying the water phase in the oil phase to prepare a water-in-oil emulsion, in some cases, homogenizing the water-in-oil emulsion, polymerizing the monomers dissolved in the water phase of the water-in-oil emulsion to obtain the polymer as a water-in-oil emulsion. If desired, a self-inverting surfactant can be added after the polymerization is complete in order to obtain the water-in-oil self-inverting emulsion.

The oil phase may include one or more inert hydrophobic liquids. Examples of suitable hydrophobic liquids include aliphatic and aromatic hydrocarbon liquids such as, e.g., benzene, xylene, toluene, paraffin oil, mineral spirits, kerosene, naphtha, and the like. In some embodiments, the oil phase includes a paraffinic oil.

Water-in-oil emulsifying agents may be used for preparing the emulsion polymers useful in the method of the invention. Suitable emulsifying agents include sorbitan esters of fatty acids, ethoxylated sorbitan esters of fatty acids, and the like, or mixtures thereof. Preferred emulsifying agents include sorbitan monooleate, polyoxyethylene sorbitan monostearate, polyoxyethylene sorbitan monolaurate, and the like. The sorbitan can be substituted with sucrose, glycol, glycerin, and the like. Additional details on these agents may be found in McCutcheon's Detergents and Emulsifiers, North American Edition, 1980. Any inverting surfactant or inverting surfactant mixture described in the prior art may be used. The amount of emulsifying agent utilized may be varied in order to optimize polymer make down and also to improve separation and recovery of the fats oil and greases present in the process stream. While the use of latex flocculants may be preferred in some embodiments, it is also possible to feed one or more anionic flocculants, alone or in combination, with an additional point source feed of one of the surfactants in order to facilitate and/or optimize separation and recovery of oil from the float layer. Representative inverting surfactants include, e.g., ethoxylated nonylphenol, ethoxylated linear alcohols, and the like, and combinations thereof. In some embodiments, the inverting surfactant includes one or more ethoxylated linear alcohols.

Upon flocculant addition to, e.g., dry milling stillage process streams, these same emulsifying agents and/or surfactants may interact with the oil, e.g., corn oil, which is bound to the surfaces of the solid constituents of the stillage, or the emulsifying agents and/or surfactants may interact with the unattached oil present in these dry milling streams. This interaction enables the oil, e.g., corn oil, to break free from the solid surfaces and be removed by separation processes such as high speed centrifugation. These same surface active chemicals also may help emulsify unattached oil preventing attachment to solid material present in the stillage process streams, which also aids in the removal of oil from the stillage.

Dispersion polymers may be prepared by combining water, one or more inorganic salts, one or more water-soluble monomers, any polymerization additives such as chelants, pH buffers or chain transfer agents, and a water-soluble stabilizer polymer. Examples of suitable dispersion polymers and methods of preparing them may be found in U.S. Pat. No. 9,776,105. The advantages of preparing water-soluble polymers as water continuous dispersions are similar to those provided by inverse emulsion polymers. The water continuous dispersion polymers have the further advantages in that they contain no hydrocarbon oil or surfactants, and require no surfactant for “inversion” or activation.

Dry polymers suitable for use in the method of the invention include those described in U.S. Pat. No. 9,776,105.

In some embodiments, an anionic polymer is used which has an anionic charge of from about 10 to about 100 mole percent, e.g., from about 30 to about 70 mole percent, and more particularly with an anionic charge of about 35 to about 45 mole percent. In some embodiments of this invention, the anionic polymer includes an acrylamide-acrylic acid sodium salt copolymer, an acrylamide-2-acrylamido-2-methyl-1-propanesulfonic acid sodium salt copolymer, or a combination thereof. Examples of suitable anionic polymers include acrylamide-acrylic acid sodium salt copolymers and acrylamide-2-acrylamido-2-methyl-1-propanesulfonic acid sodium salt copolymer, one or both having a 25 anionic charge of about 10 to about 90 mole percent, and any combination thereof.

Emulsion polymers may be inverted as a 0.1 to 5.0 percent by weight solution in clean water according to standard practices for inverting latex flocculants as described herein. The polymer may be applied to the stillage or slop process stream. Dry anionic polymer flocculants may be used in a similar fashion with the product being made up at concentrations of 0.1 to 1.5 percent polymer product according to the standard practices and recommended polymer aging times for preparing dry flocculants.

In some embodiments, the anionic flocculant includes a polymer comprising a monomer unit derived from a monomer selected from 2-acrylamido-2-methylpropane sulfonic acid (“AMPS”), 2-acrylamido-2-methylbutane sulfonic acid (“AMBS”), [2-methyl-2-[(1-oxo-2-propenyl)amino]propyl]-phosphonic acid, methacrylic acid, acrylic acid, salts thereof, and combinations thereof. In some embodiments, the anionic flocculant is a polymer comprising a monomer unit derived from acrylic acid. An exemplary anionic flocculant includes GR-109, a high molecular weight inverse-phase emulsion consisting of ˜25% polymer solids of polyacrylamide/acrylate and marketed commercially by Nalco Company, Naperville, Ill.

The effective dosage, addition point(s) and mode of addition of anionic polymer to the stillage process stream may be empirically determined to obtain a desired polymer/particle interaction and optimize the chemical treatment program performance. Generally, the anionic polymer may be added to the stillage process stream at a dosage providing a final concentration of the anionic polymer in the stillage of about 10 to about 1,000 ppm, e.g., at a dosage of about 50 ppm to about 1,000 ppm, at a dosage of about 100 ppm to about 1,000 ppm, at a dosage of about 200 ppm to about 1,000 ppm, at a dosage of about 500 ppm to about 1,000 ppm, at a dosage of about 50 ppm to about 100 ppm, or at a dosage of about 50 ppm to about 500 ppm. In some embodiments, the anionic polymer is added to the stillage in an amount sufficient to provide a concentration of anionic polymer in the stillage of from about 50 ppm to about 500 ppm.

In some embodiments, the coagulant and/or flocculant used is GRAS approved, meaning it satisfies the requirements for the United States' FDA category of compounds that are “Generally Recognized as Safe.” Using coagulants and/or flocculants that are GRAS approved is advantageous in that they need not be removed in certain applications, and can be included in the distiller grains and be fed to livestock and/or other animals, when used within the dosage and application guidelines established for the particular product formulation.

In some embodiments, the method of the present invention produces a two phase product, wherein one phase is rich in solids such as proteins and one is predominantly water. In at least one other embodiment, the method of the invention produces a three phase product, wherein one phase is rich in insoluble materials such as solids and/or proteins, one is predominantly water, and one is predominantly oil. The formation of a free-standing oil layer may vastly reduce the cost of otherwise removing oil from either of the water or, in particular, the insoluble material phases.

In some embodiments, the method of the present invention reduces the energy required to process whole stillage, thin stillage, concentrated thin stillage and/or syrup by reducing the amount of suspended solids present within the stillage. Suspended solids distribute mass throughout the stillage and when the stillage undergoes shear forces in separation equipment, the suspended solids significantly increase the energy required to properly separate the suspended solids and remove water from the stillage. The method of the present invention accordingly reduces the energy required in the solids separation steps of any of the de-watering processes including centrifugation or filtration, and reduces the amount of energy required for removing water during concentration or evaporation. Thus, the method of the present invention allows an ethanol processing facility to process more stillage without additional energy or to process stillage faster without additional energy by reducing the shear energy requirements and improving unit operation and process efficiency when the suspended solids are removed from the stillage.

The method of the invention also advantageously allows the composition of the backset to be changed favorably by removing the suspended solids. In conventional methods, it is difficult to remove certain solid materials because they remain suspended in the stillage and return to the front of the plant within the backset. Industry tends to re-use backset because it allows otherwise escaped materials to be recaptured on subsequent processing. Also, backset liquid reduces the need for additional fresh water thus lowering water costs. However, suspended materials contained therein continually increase in concentration each time the backset is recaptured and, as a result, shear energy requirement perpetually increases. By removing suspended solids in accordance with the present invention, water savings can be achieved, solids do not escape, and shear forces do not invariably rise. By improving quality of the backset, the method of the present invention increases production yield, improves evaporator performance/efficiency, reduces evaporator fouling and increases evaporator throughput.

In some embodiments, the method of the present invention reduces the energy requirements of the system by reducing the energy needed to concentrate the stillage. In other embodiments, by improving the quality of the backset, the method of the present invention may facilitate and/or increase the efficiency of ethanol production. In yet other embodiments, the flocculant and coagulant facilitate the increased recovery of grain solids and oil, e.g. corn oil.

In the method of the invention, the treated stillage is clarified via a solid/liquid separation process, to produce a clarified stillage comprising a clarified aqueous phase and a separated solids phase, e.g., which may be in the form of a float layer. In accordance with the present invention, the treated stillage is clarified upstream, i.e., prior to, a separation, concentration, and/or evaporation step used in conventional processing of stillage from ethanol production. In at least one aspect of this invention, one or more microparticulate settling aids may be added to the stillage process stream. The stillage may be aged for a relatively short period of time (0.5 to about 10 hours). “Aged” refers to the time that the stillage is left to sit in contact with one or more aids before heat and pressure are applied to this stillage mixture.

Separation of the water from the coagulated and flocculated stillage solids may be accomplished using any means commonly used for solid/liquid separation, such as a settling tank. In at least one embodiment, the stillage solids, fats and oils are concentrated and recovered on a float layer using a DAF (dissolved air flotation unit), IAF (induced air flotation unit) or GEM (gas energy mixing unit). Other embodiments contemplated by this invention include the removal of stillage solids by other sold/liquid separation devices such as a centrifuge, a recessed chamber filter press, rotary drum vacuum filters, belt presses, vacuum filters, pressure filters or membrane filtration.

In some embodiments, the float layer produced according to the method of the invention comprises a higher protein content relative to a float layer produced by conventional methods that utilize a flocculant in the absence of a coagulant. In some embodiments, the total protein recovery into the settled solid phase (simulating a DAF float) obtained according to the present invention under laboratory conditions (e.g., at approximately room temperature) is at least about 5 wt. % greater, e.g., at least about 10 wt. % greater, e.g., at least about 15 wt. % greater, e.g., at least about 20 wt. % greater, e.g., at least about 30 wt. % greater, than the total protein recovery into the settled solid phase obtained by conventional methods that utilize a flocculant in the absence of a coagulant. In other embodiments, the total protein recovery into the float layer obtained in accordance with the present invention under the operating conditions of a typical ethanol biofuel plant, which typically operate at temperatures of about 180° F. to about 200° F., is at least about 1 wt. % greater, e.g., at least about 2 wt. % greater, e.g., at least about 3 wt. % greater, e.g., at least about 4 wt. % greater, e.g., at least about 5 wt. % greater, than the total protein recovery into a float layer obtained by conventional methods that utilize a flocculant in the absence of a coagulant.

In some embodiments of the present invention, the stillage processing further includes separating at least a portion of the oil from the separated solids phase, which may be in the form of, e.g., a float layer, to produce a de-oiled solids phase, e.g., a de-oiled float layer. Any suitable method may be used to separate at least a portion of the oil from the separated solids phase. In some embodiments, the separation process may include heating and mechanical processing. In some embodiments, the temperature applied to the aged mixture is relatively low, for example from about 150° F. to about 220° F. While using such a low temperature would not ordinarily be expected to result in high oil yields, the method of the present invention unexpectedly has been found to produce high oil yields at such temperatures. The mechanical processing may be performed using known oil/solid/water separation techniques such as decanter, tricanter and stacked disk centrifuges. In some embodiments, the mechanical processing is performed with a stacked disk centrifuge.

In some embodiments, an aid is used to recover oil from the stillage by forming different phase layers. Suitable oil recovery aids may include water-in-oil emulsifying agents conventionally used as oil recovery aids such as, e.g., sorbitan esters of fatty acids, ethoxylated sorbitan esters of fatty acids, and the like, and mixtures thereof. Examples of suitable emulsifying agents include sorbitan monooleate, polyoxyethylene sorbitan monostearate, polyoxyethylene sorbitan monolaurate, and the like. The sorbitan may be substituted, e.g., with sucrose, glycol, glycerin, and the like. Examples of suitable agents may be found in McCutcheon's Detergents and Emulsifiers, North American Edition, 1980. The amount of emulsifying agent utilized may be varied in order to optimize polymer make down and also to improve separation and recovery of the fats, oil, and/or greases present in the process stream. In some embodiments, a conventional inverting surfactant or inverting surfactant mixture may be used. Representative inverting surfactants may include, e.g., ethoxylated nonylphenol, ethoxylated linear alcohols, and the like. In some embodiments, one or more ethoxylated linear alcohols are used.

In some embodiments, the oil recovery agents may include one or more surfactants such as, e.g., a propylene glycol ester, a polyglycol ester, a polyglycerol fatty ester blend, a polyglycerol oleate ester, a block copolymer of ethylene oxide-propylene oxide polymer, a vegetable oil, a vegetable oil ethoxylate, and combinations thereof.

In some embodiments, the oil recovery agent includes hydrophobic or hydrophilic silica compounds. In some embodiments, the oil recovery agent includes propylene glycol.

In some embodiments, the oil recovery agent is a combination or blend of two or more of the surfactants, and/or emulsifying agents, and/or other recovery agents described herein.

In at least one embodiment, the oil recovery agent includes at least one surfactant and at least one microparticulate comprising hydrophilic silica, and the like, e.g., as described in U.S. Patent Application Publication No. 2018/0273878. In at least one embodiment, the oil recovery agent includes a composition available from Nalco Company, Naperville, Ill., such as, for example, a composition containing a propylene glycol ester, a hydrophobic silica, a polyglycol ester, a polyglycerol oleate ester, a polyethoxylate sorbitan, a polyethoxylate sorbitan ester, and the like, and combinations thereof, e.g., as described in U.S. Patent Application Publication No. 2018/0071657.

Suitable oil recovery agents also may include, for example, oil separation aids supplied by Applied Material Solutions (“AMS”) of Elkhorn, Wis. (United States). Examples of suitable oil recovery agents may include oil separation aids supplied by AMS under product numbers 8111, 8112, and 8113. Suitable oil recovery agents may include, for example, compositions containing a blend of 75-95% polysorbate 80, 5-15% AMS hydrophobic precipitated silica, and ≤10% petroleum hydrocarbon; compositions containing a blend of 75-95% castor oil ethoxylate, 5-15% AMS hydrophobic precipitated silica, 10-30% vegetable oil, and ≤10% propylene glycol; and compositions containing a blend of 75-95% polysorbate 80, 5-15% AMS hydrophobic precipitated silica, and ≤10% PEG ester blend. In some embodiments, the oil recovery agent includes a blend of 75-95% castor oil ethoxylate which includes polyoxyl 35 castor oil or which includes a mixture of polyethylene glycol (polyoxyethylene) castor oil compounds containing from 2 to about 2000 ethylene glycol (oxyethylene) units, 5-15% AMS hydrophobic precipitated silica which includes polydimethylsiloxane treated silica or siliconized silica, 10-30% vegetable oil, and ≤10% propylene glycol.

The oil recovery agent preferably interacts with the oil, e.g., corn oil, which is either bound to the surfaces of the solid constituents of the stillage, or with the unattached oil present in these dry milling streams. This interaction enables the oil to break free from the solid surfaces and be removed by separation process such as high speed centrifugation. These same surface active chemicals also may help to emulsify unattached oil preventing attachment to solid material present in the stillage process streams which also aids in the removal of oil from the stillage.

Generally, the oil recovery agent may be added to the stillage process stream at a dosage sufficient to provide a final concentration of oil recovery agent in the stillage of from about 10 ppm to about 1,000 ppm, e.g., from about 50 ppm to about 1,000 ppm, from about 100 ppm to about 1,000 ppm, from about 200 ppm to about 1,000 ppm, from about 500 ppm to about 1,000 ppm, from about 50 ppm to about 100 ppm, or from about 50 ppm to about 500 ppm of oil recovery agent in the stillage.

In some embodiments, the method of the invention includes treating and/or drying a de-oiled separated solids phase, which in some embodiments may be in the form of a de-oiled float layer, to produce distiller dry grains comprising the protein. Using methods known in the art, the de-oiled solids phase (e.g., de-oiled float layer) may be subjected to mechanical processes to remove non-protein dry mass prior to drying.

The method of the invention improves the total protein content of the distiller dry grain.

The present invention also provides a composition comprising the dried/dry separated solids phase produced according to method of the invention, e.g., a composition comprising the distiller dry grain produced according to the method of the invention. The invention further provides a nutritional product comprising the dried/dry separated solids phase produced according to method of the invention, e.g., a nutritional product comprising the distiller dry grain produced according to the method of the invention. The invention moreover provides a livestock feed or fertilizer comprising the dried/dry separated solids phase produced according to method of the invention, e.g., livestock feed or fertilizer comprising the distiller dry grain produced according to the method of the invention.

The following examples further illustrate the invention but, of course, should not be construed as in any way limiting its scope.

### Example 1

This example demonstrates typical protein recovery from thin stillage in a dry grind ethanol plant, after treatment of the thin stillage with anionic flocculant in the GEM process, as in FIG. 1.

TABLE 1
GEM Protein Recovery from Thin Stillage in an Ethanol Plant

| | GEM Protein |
|---|---|
| | Recovery |
| Experiment 1 | 40.90% |
| Experiment 2 | 38.30% |
| Experiment 3 | 31.10% |
| Average | 36.77% |
| Stand. deviation | 5.08% |

GEM protein concentration was measured using a combustion method (AOAC 990.03). Protein recovery was calculated as: ((% protein in thin stillage-% protein in GEM effluent)/% protein in thin stillage) %.

This reference example demonstrates that, on average, less than 40% of possible protein was recovered from thin stillage after prior art treatment with anionic flocculant in the GEM process.

### Example 2

This example demonstrates the percentage of moisture, dry matter, protein and fat recovered in samples from along the process stream collected in the ethanol plant of Example 1.

TABLE 2
Percentage of Various Components Recovered in Samples
From Along the Process Stream in an Ethanol Plant

| Plant samples | Moisture % | Dry Matter % | Protein % | Fat % |
|---|---|---|---|---|
| Thin Stillage | 93.45 | 6.55 | 2.1 | 1.8 |
| GEM Float | 78.75 | 21.25 | 5.9 | 8.8 |
| De-oiled float | 83.21 | 16.79 | 5.9 | 3.8 |
| Dry de-oiled float | 10 | 90 | 31.6 | 20.6 |
| (calculated) | | | | |
| 100% oil removed | 10 | 90 | 41.0 | 0.0 |
| DDGS | | | | |
| (calculated) | | | | |

Protein % for samples of thin stillage, GEM float and de-oiled float are measured using a combustion method (AOAC 990.03). Fat % was measured using an acid hydrolysis method: AOAC 954.04. Percentage moisture was measured using a vacuum oven method (AOAC 969.35). The last two rows represent the theoretical calculation of protein content in the DDGS if one assumed that 10% moisture was left in the sample and 100% of the oil had been removed from the dry de-oiled float.

This reference example demonstrates that, at ˜40% protein recovery in the GEM float (see Table 1), even if all the oil could subsequently be removed, the final protein concentration in the DDGS is only 41% at 10% moisture, in the absence of any additional method to remove non-protein dry mass.

### Example 3

This example demonstrates the total percent protein recovery from thin stillage treated with anionic flocculent and different coagulants.

Unlike the field testing performed in previous examples, the present example represents laboratory data gathered using a jar test. The procedure was as follows: first, a sample of untreated room temperature thin stillage was poured into a beaker (e.g., 500 mL). While mixing at 200 rpm, coagulant was added (or nothing was added if sample was only treated with flocculant or a filter), and the sample was mixed for 60 sec. The mixing rate was lowered to 100 rpm and anionic flocculant solution (at 30 ppm active) was added and mixed for 30 sec., followed by slow mixing at 50 rpm for 2 mins. Mixing was stopped, and the sample allowed to settle for 30 mins. Finally, the supernatant was removed, and the turbidity and protein concentrations were measured.

The total percent protein recovery in the settled solid phase which simulates the GEM float in the field was determined after samples of thin stillage were treated with a fixed amount of the same anionic flocculant and the indicated coagulant. Organic nitrogen was used to represent the amount of protein in the samples. Organic nitrogen=Total Kjeldahl Nitrogen (TKN, U.S. EPA method 351.2R2.0)—Ammonia (U.S. EPA method 350.1R2.0). Total percent protein recovery was calculated as: 100%—(amount of organic nitrogen in supernatant of each treated sample/amount of organic nitrogen in untreated thin stillage sample) %.

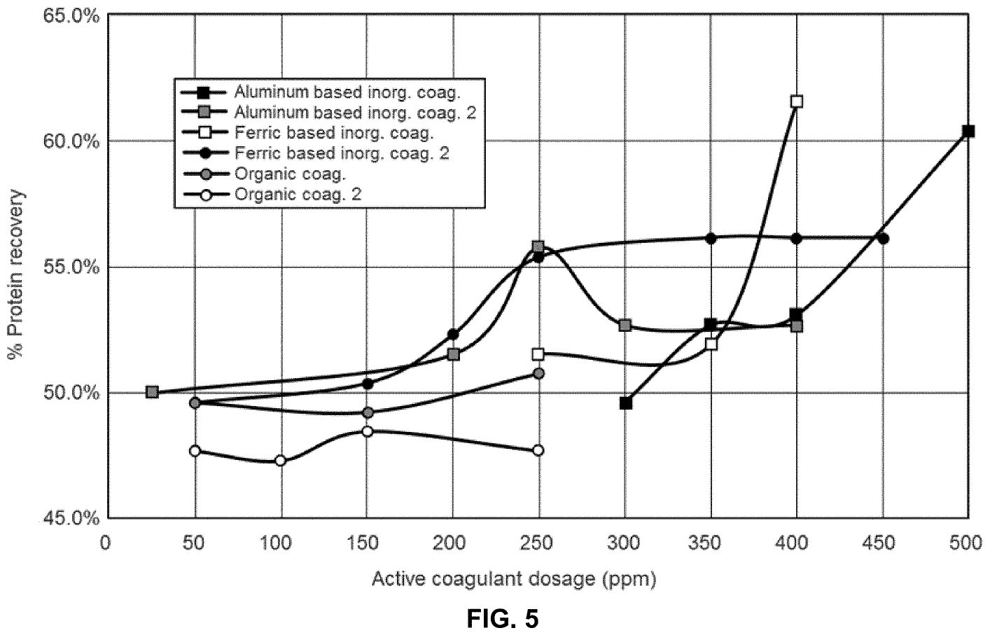

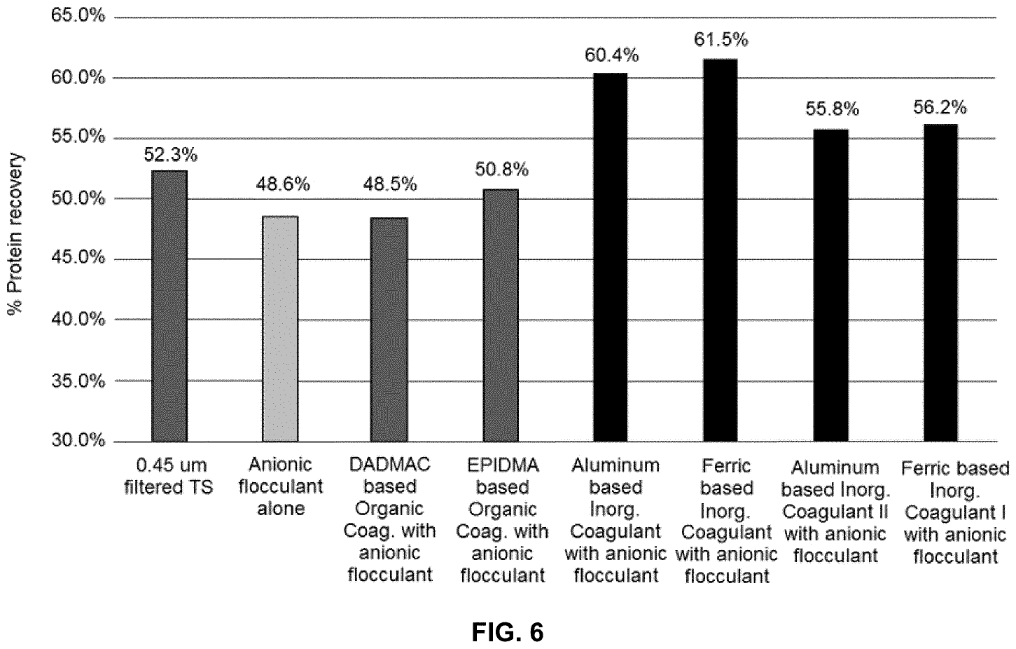

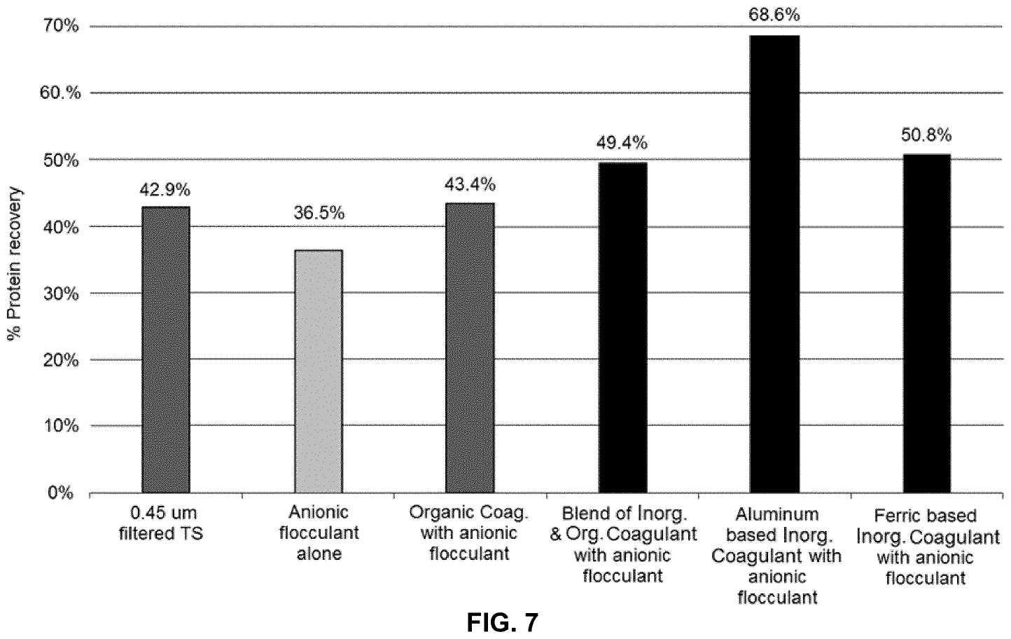

The amount of flocculant was fixed for all jar tests, while the amount of coagulant was varied for each coagulant chemistry to obtain a dosage curve. For each coagulant, a dosage curve was prepared, with a representative example shown in the graph in FIG. 5. The maximum protein recovery in the settled solid phase obtained with each coagulant depicted in FIG. 5 was determined, and the results are shown in the bar graphs 1 in FIG. 6. The graph in FIG. 7 depicts the protein recovery in the settled solid phase obtained with each coagulant in a comparable experiment.

As depicted in Graphs 6 and 7, the percent protein recovery in the lab experiment for the thin stillage treated with anionic flocculant alone is consistent with the field data depicted in reference Table 1. The data in Graphs 6 and 7 also show that about 40% to 50% of the protein present in the thin stillage has a particle size larger than 0.45 μm (demonstrated by capture by a 0.45 μm filter). Flocculant treatment alone does not capture all of that protein into the GEM float, thus allowing the rest of the protein (>50%) to be lost in the effluent. Treatment with organic coagulant in addition to anionic flocculant demonstrates little improvement in protein recovery over the use of flocculent by itself.

Unexpectedly, addition of inorganic coagulant was found to be very effective in recovering the proteins that are smaller than 0.45 μm and significantly increased the percent protein recovery in the settled solid phase (which can be as high as 68%, as shown in the graph in FIG. 7), which the organic coagulant does not do.

### Example 4

This example demonstrates the total percent protein recovery from thin stillage treated with anionic flocculent and inorganic coagulant in a field trial.

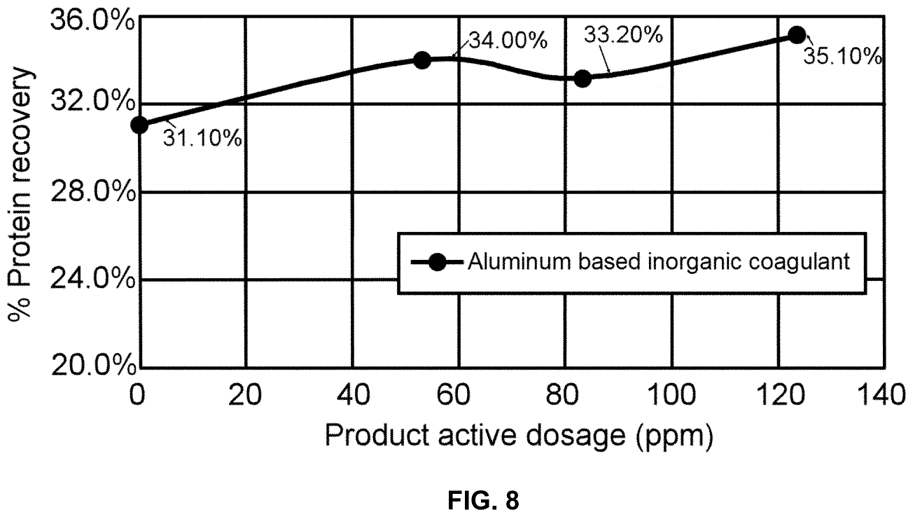

The field trial was conducted in a dry grind ethanol plant in Illinois. The indicated coagulant at the indicated amount was mixed inline with the thin stillage stream at the plant operating temperature of 180° F. to 200° F., followed by anionic flocculant at a fixed dosage of 40 ppm before the stream traveled into the GEM unit. Both thin stillage and GEM float samples were taken after each coagulant/flocculant dosage. Dry weight protein concentration and total dry mass were determined as in Example 2. The graph depicted in FIG. 8 shows the dosage curve, prepared as described for FIG. 5, for the aluminum based inorganic coagulant used in the field trial. Total protein % recovery in the field trial was calculated as: (flow of GEM float)*(protein % dry weight in GEM float)/(flow of thin stillage)*(protein % dry weight in thin stillage.

FIG. 8 shows that same phenomenon demonstrated in the lab was also observed in the field. Addition of inorganic coagulant to the anionic flocculant treated sample was found to increase total protein recovery in the GEM float compared to treatment with anionic flocculant alone (active dosage at zero coagulant added). The use of inorganic coagulant in combination with an anionic flocculant unexpectedly was found to further increase the protein recovery in the GEM float under operating conditions used in ethanol plants. Thus the final DDGS product will provide higher value animal feed products.

### Example 5

This example demonstrates the percent oil recovery to solid phase, and the turbidity of the liquid phase, of a thin stillage sample treated with a fixed amount of the same anionic flocculant and with, or without, inorganic coagulants.

TABLE 3
Percent Oil Recovery to the Solid Phase and Remaining
Turbidity of the Liquid Phase in a Process Stream
Treated with Various Additives

| | % Oil recovery | Turbidity of |
|---|---|---|
| | to solid phase | liquid phase (NTU) |
| Anionic flocculant alone | 83.40% | 290 |
| Ferric based inorg. coagulant with | >94% | 105 |
| anionic flocculant | | |
| Aluminum based inorg. coagulant | >94% | 93.5 |
| with anionic flocculant | | |

The present example represents laboratory data generated using the jar test, with the test conducted and the data calculated as in Example 3.

As shown in Table 3, the addition of inorganic coagulant to the processing of thin stillage can also improve the capture of oil to the solid phase and reduce the turbidity of the clarified thin stillage that continues on in the process, either to an evaporator to form syrup or back to the cook process as backset. The increase of oil capture to the solid phase can help to recover more corn oil, which can be used as biodiesel. Further, the improvement in the clarity of the clarified thin stillage (i.e., enhanced removal of insoluble components) can help to improve the backset quality, which in turn: increases ethanol plant production yield, improves evaporator performance/efficiency, reduces evaporator fouling, and increases evaporator throughput.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of processing stillage from an ethanol production process, the method comprising:
treating stillage comprising oil, protein, and water upstream of a separation, concentration or evaporation step with at least one coagulant and at least one flocculant, to produce a treated stillage comprising solids which include at least a portion of the oil and protein; and
subjecting the treated stillage to a solid/liquid separation process, to produce a clarified stillage comprising a clarified aqueous phase and a separated solids phase, wherein the separated solids phase comprises at least a portion of the solids from the treated stillage.

2. The method of claim 1, wherein the stillage is whole stillage or thin stillage and the separated solids phase is in the form of a float layer.

3. The method of claim 1, wherein the stillage is thin stillage and the separated solids phase is in the form of a float layer.

4. The method of claim 3, comprising treating the thin stillage with the at least one coagulant and at least one flocculant upstream of a concentration or evaporation step, to produce a treated thin stillage.

5. The method of claim 4, further comprising separating at least a portion of the oil from the float to produce a de-oiled float.

6. The method of claim 2, further comprising drying the de-oiled float to produce distiller dry grain comprising the protein.

7. The method of claim 1, wherein the at least one coagulant comprises one or more inorganic coagulants or a blend of one or more inorganic coagulants and one or more organic coagulants.

8. The method of claim 7, wherein the one or more inorganic coagulants comprises aluminum sulfate, poly-aluminum chloride, aluminum chlorohydrate, sodium aluminate, ferric sulfate, ferric chloride, or ferrous sulfate, or a combination thereof.

9. The method of claim 7, wherein the one or more inorganic coagulants comprises aluminum, iron, or a combination thereof.

10. The method of claim 7, wherein the one or more organic coagulants comprises poly(diallyldimethylammonium chloride) (polyDADMAC), epichlorohydrin-diethylamine, dimethylamine, polyamines, polyquaternary amines, or a combination thereof.

11. The method of claim 10, wherein the one or more organic coagulants comprises polydiallyldimethylammonium chloride (polyDADMAC), epichlorohydrin-dimethylamine, or a combination thereof.

12. The method of any claim 1, wherein the at least one flocculant comprises an anionic flocculant.

13. The method of claim 12, wherein the anionic flocculant is a polymer comprising a monomer unit derived from a monomer selected from 2-acrylamido-2-methylpropane sulfonic acid (“AMPS”), 2-acrylamido-2-methylbutane sulfonic acid (“AMBS”), [2-methyl-2-[(1-oxo-2-propenyl)amino]propyl]-phosphonic acid, methacrylic acid, acrylic acid, salts thereof, and combinations thereof.

14. The method of claim 12, wherein the anionic flocculant is a polymer comprising a monomer unit derived from acrylic acid.

15. The method of claim 5, wherein separating at least a portion of the oil from the float layer comprises treating the float layer with an oil recovery agent, which comprises a sorbitan ester of a fatty acid, an ethoxylated sorbitan ester of a fatty acid, or a combination thereof.

16. The method of claim 15, wherein the oil recovery agent comprises polyoxyethylene sorbitan monostearate.

17. The method of claim 5, wherein separating at least a portion of the oil from the float layer comprises treating the float layer with an oil recovery agent, which comprises a propylene glycol ester, a polyglycol ester, a polyglycerol fatty ester blend, a polyglycerol oleate ester, a block copolymer of ethylene oxide-propylene oxide polymer, a vegetable oil, a vegetable oil ethoxylate, or a combination thereof.

18. The method of claim 5, wherein separating at least a portion of the oil from the float layer comprises treating the float layer with an oil recovery agent, which comprises at least one surfactant and at least one hydrophilic silica.

19. The method of claim 5, wherein separating at least a portion of the oil from the float layer comprises treating the float layer with an oil recovery agent, which comprises a blend of 75-95% polysorbate 80, 5-15% hydrophobic precipitated silica, and ≤10% petroleum hydrocarbon; a blend of 75-95% castor oil ethoxylate, 5-15% hydrophobic precipitated silica, 10-30% vegetable oil, and ≤10% propylene glycol; or a blend of 75-95% polysorbate 80, 5-15% hydrophobic precipitated silica, and ≤10% PEG ester blend.

20. The method of claim 2, wherein the solid/liquid separation process comprises dissolved air flotation, induced air flotation, gas energy mixing or a combination thereof.

21. The method of claim 5, wherein separating at least a portion of the oil from the float layer comprises heating and mechanical processing.

22. The method of claim 21, wherein the mechanical processing is performed with a decanter, a tricanter, a stacked disk centrifuge, or a combination thereof.

23. The method of claim 21, wherein the mechanical processing is performed with a stacked disk centrifuge.

24. The method of claim 21, wherein the heating produces a temperature of from about 150° F. to about 220° F.

25. An ethanol production process comprising the method of claim 1, wherein the ethanol production process is an ethanol biofuel process, a spirits distillery process, or a brewery process.

26. The ethanol production process of claim 25, wherein the ethanol production process is an ethanol biofuel process.

27. The ethanol production process of claim 26, wherein the ethanol biofuel production process is a wet milling process or a dry grind process.

28. The ethanol production process of claim 27, wherein the ethanol biofuel production process is a dry grind process.

29. The method of claim 1, further comprising drying the separated solids phase to produce dried grains.
